(19) United States
(12) Patent Application Publication
(10) Pub. No.: US 2022/0051787 A1
(43) Pub. Date: Feb. 17, 2022

(54) Computerized Risk-driven Appointment Management and Reimbursement Optimization

(71) Applicant: LumiSight, Inc., Honolulu, HI (US)

(72) Inventors: Bryson Sugai, Honolulu, HI (US); Catherine Higuchi, Honolulu, HI (US)

(21) Appl. No.: 16/995,473

(22) Filed: Aug. 17, 2020

(51) Int. Cl. G16H 40/20; G16H 50/30; G16H 10/60; G16H 70/20; G16H 70/60; G16H 50/20; G06Q 10/06; G16H 50/50; G16H 50/70; G06N 5/02; A61B 5/00

(52) U.S. Cl. CPC G16H 40/20; G16H 50/30; G16H 10/60; G16H 70/20; G16H 70/60; G16H 50/20; G06Q 50/26; G16H 50/50; G16H 50/70; G06N 5/025; A61B 5/4842; A61B 5/7275; A61B 5/746; G06Q 10/06316

(57) ABSTRACT

Disclosed is a system for optimizing risk scores of patients in a patient panel and prioritizing appointments based on the optimized risk scores of each patient subscribed to the service provider. After assigning an initial risk score, the risk score of each patient is optimized by determining any discrepancy between a risk score determined by a rules engine and each patient's assigned risk score, and assigning a patient an optimized risk score, which includes the risk score determined by the rules engine where such discrepancy is determined or the patient's assigned risk score where no such discrepancy is determined. Appointments can be prioritized in accordance with the risk scores for the patient.

### BACKGROUND

Beginning in 2014 under the Affordable Care Act (ACA, also known as “ObamaCare”) the health insurance industry shifted from the traditional risk-based insurance model to one that is community-rated and guaranteed issue. All enrollees must be accepted and pricing may not vary based on health status. Due to these changes in the insurance model for the beneficiaries, various government programs were implemented to absorb some of the newly added risk. A risk adjustment program was implemented that transfers payments and charges between health insurance carriers within a risk pool based on the relative riskiness of a population. This program was intended to dampen the effects of adverse selection. As an example, because insurers were no longer allowed to deny coverage or charge increased rates for many preexisting conditions, the risk adjustment program reimburses those insurers who accept individuals (or groups) that are deemed to be “riskier.” The reimbursements are derived from the insurers who do not take the “riskier” individuals or groups, so that insurers who do not accept riskier clients will not receive a windfall.

ICD-10 disease coding was supplemented with Hierarchical Condition Categories (HCC) codes indicating relative seriousness of the condition. Hierarchical condition category coding helps communicate patient complexity and paint a picture of the whole patient. In addition to helping predict health care resource utilization, a risk adjustment factor (RAF) score is assigned, based on the HCC, to risk adjust quality and cost metrics. By accounting for difference in patient complexity, quality and cost, performance can be more appropriately measured. An HCC code including a Major Complications or Comorbidities (MCC/CC) designation would further indicate a higher RAF is appropriate.

Insurance carriers can use a patient's RAF score to predict costs. For example, a patient with few serious health conditions could be expected to have lower average medical costs for a given time than a patient with multiple chronic conditions. Physicians are often reimbursed on a per patient per month basis, with the reimbursement rate based on the RAF score (as well as other factors) for their entire designated patient panel.

It is also important for providers to provide the appropriate level of care to patients and encourage healthy patient engagement. The provider's services can be ineffectively utilized by visits from patients who are relatively healthy and don't necessarily need to see a physician, leaving a high-risk patient who needs a visit unable to schedule an appointment.

What is needed is a system to aid health care practitioners in providing the right level of care for patients, effectively utilizing their resources, and accurately reflecting patient risk to payers to optimize their reimbursement to allowable limits.

### SUMMARY

Connecting rules engines, databases, and communication interfaces, e.g., by a software agent, is provided which has the following objectives and features:
A. Optimizing provider's future patient appointments (interaction opportunities) for each patient, based on prioritization of high-risk patient appointments;
B. Calculating ICD-10, HCC and RAF (for both clinical and/or billing/claims) and adjustment thereof, to provide accurate RAF coding for particular diagnosis by the provider, with discrepancy list and review items signals, where likely understatement of risk and where indicated, reviews or signals provider of the likely understatement, are determined, e.g., by a review software agent. The need for adjustment is alerted, for example, where there is a difference between the physician assigned RAF (“recorded RAF”) and the RAF in the claim to the insurer (“claim risk”).
C. Using e.g., a rules engine to categorize and then threshold patient risk adjustment factor (RAF) score determined from HCC codes; where the thresholding is used (e.g., by the software agent) so that appointments for higher risk patients are prioritized to increase needed interactions for such higher risk patient and to reduce overall usage of medical resources (i.e., physician and care team's capacity within their normal working hours); by de-prioritizing appointments for lower risk patients.
D. Optimizing scheduling and optimizing resource utilization by determining (i) optimal patient number; (ii) optimal visit schedule for each patient; and (iii) maximum provider capacity to turn over patients, based on actual averages over time combined with recommended visit times based on RAF scores; all of which is determined e.g., by the software agent.
E. Optimizing (based on status) and predicting (based on patient ICD-10 codes, including HCC codes, and RAF) future appointments for patients, by, using data analytics and AI to analyze the physician's patient panel by RAF and patient engagement behavior, and to identify opportunities for the physician to improve performance, resource utilization, optimize workflows by increasing workflow efficiency and improve coding practices and maximize reimbursement e.g., by the software agent.
F. Displaying a “dashboard” showing: the provider RAFs and possibilities for adjustment of RAF for a particular patient based on Step A analysis; the RAF and engagement behavior of the physician's patient panel; the appointment schedule of each member of the panel of providers (within the entire clinic) with open appointment times shown; visualizations of each patient's scheduled, recommended and completed appointments; all of which can be searched and filtered; performed reimbursement e.g., by the software agent.
G. Fully searchable and filterable patient panel table allows for care team follow-up and management, including the care team taking action on appointment opportunities or rescheduling, based on the recommendations from data analytics and/or AI analysis in Step E, and revising incorrect RAF assignment; and providing patient contact information; and providing for determination of effectiveness of patient follow-up by the care team in increasing patient interaction/appointments e.g., by the software agent.

### BRIEF DESCRIPTION OF THE DRAWINGS

FIG. 1 illustrates connecting rules engines, databases, and communication interfaces for performing the method steps outlined in the flow charts of the specification; including the steps which can be performed in the cloud or by e.g., the software agent.

### DETAILED DESCRIPTION

Referring to the Summary, the objective of the computer-implemented method is to prioritize future patient appointments (interaction opportunities) for high risk patients, and to properly identify the RAF for each patient and correct RAFs where appropriate. In addition to the objectives and features set forth in the Summary some additional process steps and objectives and features are outlined below.

Initially: 1: An initial risk score for the patient is determined based on the diagnosis from patient history, test results, and physical and verbal examination, based on the most recent care visit with the patient.

2: The need for RAF adjustment is alerted where the entire spectrum of a patient's diagnosis is not included in the ICD-10 codes assigned or where there are significant co-morbidities, warranting an adjustment to the ICD-10 code or the RAF; or where there is a discrepancy between the assigned RAF and a risk score determined by a rules engine in the system.

3: Insurance carriers use a patient's RAF score to predict costs. For example, a patient with few serious health conditions could be expected to have average medical costs for a given time lower than a patient with multiple chronic conditions, who would be expected to have higher health care utilization and costs. Setting thresholds that meet the physician's specifications allow future appointments to be prioritized for higher risk assessments, and to reduce high usage of health care services by lower risk patients and increase the needed patient interactions, in view of the limitations of capacity

4: To optimize scheduling and optimize resource utilization, the system can optionally display past appointment data/behavior for each patient, and alert over-utilization of services and recommended additional appointments for a patient.

5: Similarly to part 4, a “dashboard” is displayed focused more on analytics at the clinic/practice level to give the practice's administrators an in-depth look at how they are performing and to better assess whether changes are needed to improve staff resourcing, physician compensation/incentives, and insurance payer contracts/agreements. The dashboard includes metrics and data visualizations on patient panel growth and attrition, risk scoring tracking and risk gap closure, patient visitation performance and distribution (including trends, in person vs. telehealth). Risk gap closure is needed where the recorded RAF is different from the claim risk.

The software agent will also offer a means for determining what changes to the panel RAF would be needed to reach a specific RAF goal for each patient or a goal average RAF for the entire panel.

The “calculator” will incorporate factors such as current average risk score, risk goal set for the physician, visit capacity for physician, and existing vs new patients impacted. It will produce the benchmarks necessary to achieve their risk goal along with recommendations on how to achieve those benchmarks, including with the tracking done by e.g., the software agent.

Providing notifications or alerts at the point-of-care, which are pertinent to the objectives, for example, “patient has HCC codes missing from or incorrectly assigned to their claims”, “Add as a possible new diagnosis and include its HCC code”, “it's been (time) since the last visit, and the applicable HCC code indicates visits more frequently” performed by e.g., the software agent.

6: Determination of effectiveness of patient follow-up by the care team in increasing patient interaction/appointments can be used by the outreach team or schedulers for patient follow-up to increase patient interactions and care management.

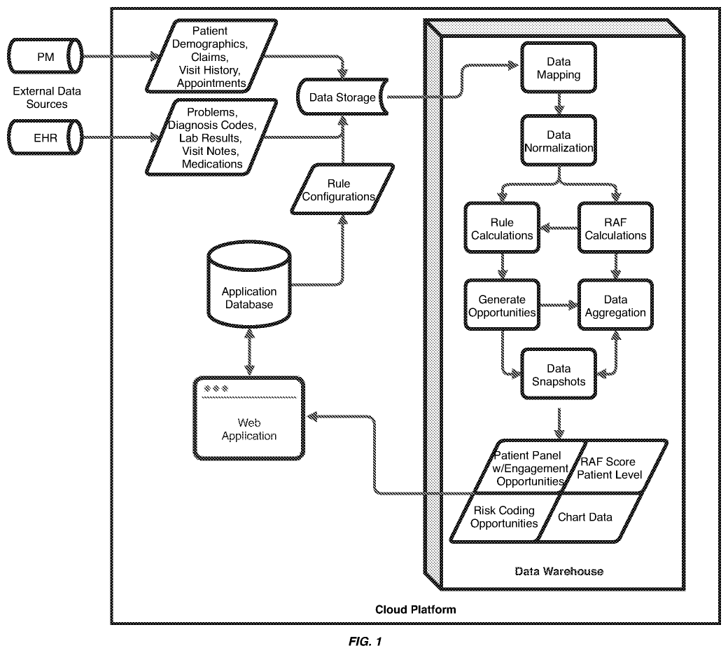

An implementation of the hardware and steps in the process to perform the features and meet the objectives outlined above, is set forth in FIG. 1, showing portions of the process which can be performed in the cloud. The flow chart below summarizes the steps taken by the system in FIG. 1. The following definitions apply for the flow chart below.

“Rule” is the rule(s) enacted to provide data configurations, where the enactment is performed in the data warehouse.

“Coding Opportunities” refers to patients with diseases or conditions which currently or in the near future are expected to warrant one or more different or additional ICD-10 diagnosis codes than those initially assigned.

“Care Utilization” refers to the extent to which the patient is utilizing the physician's care facility.

“Engagement Level” refers to the amount of engagement and interaction with the patient by the care facility and care team.

“Engagement Opportunities” refers to patient's needing or warranting a higher Engagement Level to improve their care while remaining within the engagement limits set by the RAF and/or ICD-10 or HCC codes for the patient.

Step 1: The physician assigns and assesses ICD-10 diagnosis codes (which will appear on the claims for the payers). These ICD-10 diagnosis codes are used in assessing the patient's initial risk score.

Step 2: The patient data from PM (patient profile and identification) and EHR (relating to diagnosis and treatment) are extracted, integrated and stored, preferably in cloud storage. Rule configurations from the system database are also stored there, to be used for the rules engine.

Step 3: Data from storage is transferred (preferably to a data warehouse) for mapping, to identify the source, other processing and normalization, preferably every day after business hours. Normalization produces consistent data formats and nomenclature.

Step 4: Data (preferably in the data warehouse) is processed by e.g., a rules engine using a Rule (which can be adjusted as needed). Risk Adjustment Factor (RAF) calculations are performed; where the software agent determines discrepancies between perceived risk and the assigned risk scores to identify opportunities to adjust the RAF. A higher RAF can be recommended e.g., by the software agent where there is such discrepancy; and then a data snapshot can store the RAF and risk scores at that point in time, for comparison.

Step 5: Categorize Engagement Opportunities for the patient panel and working with the RAF for individual patients, based on each patient's: Coding Opportunities, RAF, Care Utilization, and Engagement Level can be done, e.g. by the software agent. For example, where discrepancies between ICD-10 or HCC codes on the patient's chart versus what was billed for on claims, where the RAF is increased, and/or where Care Utilization or Engagement Level are below or above the limits for patients with those codes and Risk Scores, such patients present an identifiable Engagement Opportunity.

Step 6: Using the Rule and RAF calculations, data is then aggregated at different levels (e.g., clinic, provider) to produce data sets (Chart Data) used for data visualizations on the dashboard.

Step 7: Data can also be snapshotted to capture specific data at certain points in time to be used for trending and historical comparisons.

Step 8: Data from the data warehouse and results from the application database are displayed on the dashboard through the web application, for the users. The web application and application database can exchange information related to rule configurations and account setups.

Step 9: Provider accesses the web application to identify Coding Opportunities and Engagement Opportunities. The Rule can be changed to process the data differently and provide different configurations.

TABLE I below is description of some Rules, and some possible configurations to adjust RAF and patient interactions.

TABLE I

| Rule Name | Rule Description | Configurable Options |
|---|---|---|
| Protein-Calorie | Patient has [any insurance] as | insurance [any insurance] - allows |
| Malnutrition | primary insurance, [18] years | multiple selections, variable to |
| | old and older with a BMI less | customer's data |
| | than [18.5] and is not already | age [18] - allows any numeric value |
| | diagnosed as protein-calorie | bmi value [18.5] - allows any numeric |
| | malnutrition (HCC21) | value |
| High Visit Count, | Patient has [any insurance] as | insurance [any insurance] - allows |
| Low Risk | primary insurance with [3] or | multiple selections, variable to |
| | more visits in the last [12] | customer's data |
| | months and a problem risk score | visits [3] - allows any numeric value |
| | less than or equal to [1.000] | lookback months [12] - allows selection |
| | | of 3, 6, 9, 12, 18, or 24 |
| | | risk score [1.000] - allows any numeric |
| | | value |
| Low Visit Count | Patient has [any insurance] as | insurance [any insurance] - allows |
| w/ PCP, High | primary insurance with less than | multiple selections, variable to |
| Risk | or equal to [4] visits with PCP in | customer's data |
| | the past [12] months, no next | visits [4] - allows any numeric value |
| | appointment, and a problem risk | lookback months [12] - allows selection |
| | score greater than or equal to | of 3, 6, 9, 12, 18, or 24 |
| | [1.500] | risk score [1.5000] - allows any numeric |
| | | value |

Via the web application, patients or the panel can be ranked based on risk scores, RAF or Engagement Opportunities To prioritize appointments for patients in order of highest to lowest RAFs, or RAFs above one or more thresholds, and/or to de-prioritize appointments for patients below one or more thresholds. The provider can set or adjust the thresholds or modify the priorities. Patients for whom an additional appointment is warranted can be identified.

Separately, others or the provider can follow-up with patients that have been in communication with their physician and schedule new appointments through an interaction log interface.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method enacted by a system comprising a software agent which includes rules engines, databases and communication interfaces, and connecting rules engines, databases and communication interfaces, wherein the system performs the following steps using the software agent, includes rules engines, databases and communication interfaces:
assigning, to each patient in a patient panel, initial risk scores based on the patient's diagnosis and on hierarchical condition categories and/or ICD-10 codes;
extracting, integrating and storing patient data, including patient diagnosis and treatment(s);
transferring stored data to a data warehouse for: mapping, to identify the data source; data processing, under a rule providing data configurations; and data normalization;
determining a risk adjustment factor for patients for whom there is a discrepancy between perceived risk and the assigned risk scores;
determining if the risk adjustment factor represents a likely understatement of perceived risk and alerting the system user of the likely understatement;
categorizing a provider's engagement opportunities with each patient, where engagement opportunities are additional patient interactions for which reimbursement from insurance or Medicare would be available, based on the patient's hierarchical condition categories and/or ICD-10 codes and risk score or risk adjustment factor;
aggregating the mapped, processed and normalized data at different levels by applying the rule and the risk adjustment factor, to produce data sets at different levels;
displaying the stored data and the data sets on a display;
prioritizing appointments for patients in order of highest to lowest engagement opportunities, or engagement opportunities above one or more thresholds, and/or de-prioritizing appointments for patients with engagement opportunities below one or more thresholds; and
determining how to change the patient panel to reach a goal risk score or risk adjustment factor average for the patient panel and determining an optimal number of patients in the patient panel to reach said goal risk score or said risk adjustment factor average.

2. The method of claim 1 wherein the engagement opportunities for each patient are further based on re-determining the patient's hierarchical condition categories and/or ICD-10 codes in view of adjusted diagnosis or disease progression.

3. The method of claim 1 wherein the engagement opportunities for each patient are further based on re-determining the patient's risk score or risk adjustment factor.

4. The method of claim 1 wherein the engagement opportunities for each patient are further based on determining the patient's provider utilization and engagement with the provider.

5. The method of claim 1 further including identifying patients for whom additional appointments are warranted.

6. The method of claim 1 further including providing a system user the ability to set or adjust the thresholds or modify the prioritization of appointments.

7. The method of claim 1 further including reviewing on hierarchical condition categories and/or ICD-10 codes and determining likely coding errors and alerting a system user of the likely errors.

8-9. (canceled)

10. The method of claim 9 wherein the determination is based on factors including patient appointment maximum capacity and rescheduling of patient appointments required to meet the goal risk score or risk adjustment factor average.

11. (canceled)

12. (canceled)

13. The method of claim 1 wherein the initial risk scores were assigned based on the most recent care visit with the patient.

14. (canceled)

15. The method of claim 1 further including providing a system user the ability to set or adjust the thresholds or modify the prioritization of appointments.

16-19. (canceled)

20. The method of claim 1 further including determining an optimal appointment schedule for each patient to reach the goal risk score or risk adjustment factor average.

21. The method of claim 1 wherein normalization produces consistent data formats and nomenclature.

22. The method of claim 1 further including snapshotting data to capture specific data at certain points in time.

23. The method of claim 1 wherein the rule can be changed to process the data differently and/or to provide different data configurations.

24. The method of claim 1 wherein the stored data can be transferred for processing.

25. The method of claim 1 further including integrating patient identifying information and information relating to the patient's diagnosis and treatment.
